(19) United States
(12) Patent Application Publication
(10) Pub. No.: US 2016/0243382 A1
(43) Pub. Date: Aug. 25, 2016

(54) APPARATUS FOR GENERATING HIGH INTENSITY FOCUSED ULTRASOUND

(71) Applicant: KORUST CO., LTD., Anyang-si (KR)

(72) Inventor: Sung-Chan JO, Seongnam-si (KR)

(73) Assignee: KORUST CO., LTD., Anyang-si (KR)

(21) Appl. No.: 14/638,314

(22) Filed: Mar. 4, 2015

(30) Foreign Application Priority Data
Feb. 24, 2015 (JP) 10-2015-0025731

(51) Int. Cl. A61N 7/00

(52) U.S. Cl. CPC A61N 7/00; A61N 2007/0091; A61B 2562/0223; A61N 2007/0065; A61N 2007/0034

(57) ABSTRACT

An apparatus for generating high intensity focused ultrasound includes: a housing which is filled with ultrasound transmitting medium; an ultrasound transducer which is movably disposed inside the housing; and a driving unit which linearly moves the ultrasound transducer.

### CROSS-REFERENCE TO RELATED APPLICATION

This application claims priority to and the benefit of Korean Patent Application No. 10-2015-0025731 filed in the Korean Intellectual Property Office on Feb. 24, 2015, the entire contents of which are incorporated herein by reference.

### TECHNICAL FIELD

The present relates to an apparatus for generating high intensity focused ultrasound which is used as medical devices or the like.

### BACKGROUND ART

A focused ultrasound therapy technology for treating the deceased part or for skin care has been introduced. Such a focused ultrasound therapy is performed using an apparatus for generating high intensity focused ultrasound using focused ultrasound transducer which focuses ultrasound to generate high intensity focused ultrasound.

In particular, an apparatus for generating high intensity focused ultrasound includes a pulse power generator which generates pulse current and an ultrasound focusing member which receives the pulse current and focuses high intensity ultrasound. The ultrasound focusing member may be electrically connected to the pulse power generator via a cable such that an operator can freely move the ultrasound focusing member.

The ultrasound focusing member includes a housing and an ultrasound transducer which is installed inside the housing. Here, the ultrasound transducer may be formed as a piezoelectric vibrator which is formed by forming a first electrode and a second electrode on both sides of a piezoelectric member, and converts electric signal applied to the first and the second electrodes to ultrasound.

In generating ultrasound using a concave piezoelectric member, ultrasound may be focused on a certain area. At this time, the position of the focused ultrasound is called a focal point. The ultrasound energy which is focuses on the focal point has an intensity distribution of an elliptical shape. The ultrasound focused in this way is used in medical devices for cancer treatment, skin wrinkle care or the like.

However, in the conventional apparatus for generating high intensity focused ultrasound, the ultrasound transducer is fixed to an inside of the housing, so an operator must move the whole of the ultrasound focusing member in order to treat plural deceased parts or a long deceased part.

### DETAILED DESCRIPTION OF THE INVENTION

### Technical Problem

The present invention has been made in an effort to provide an apparatus for generating high intensity focused ultrasound in which an ultrasound transducer can automatically move inside a housing.

The present invention has been also made in an effort to provide an apparatus for generating high intensity focused ultrasound in which a moving position of the ultrasound transducer can be detected.

### Technical Solution

An exemplary embodiment of an apparatus for generating high intensity focused ultrasound includes: a housing which is filled with ultrasound transmitting medium; an ultrasound transducer which is movably disposed inside the housing; and a driving unit which linearly moves the ultrasound transducer.

The driving unit may include: a driving motor which is provided at the outside the housing through a fixing bracket; and a direction converting part which converts a rotation movement of a shaft of the driving motor to a linear movement and provides a linear movement force to the ultrasound transducer.

The direction converting part may include: a crank arm which is shaft-connected to the driving motor; a crank pin which is provided at an edge portion of the crank arm; a linear slider which has a long slot into which the crank pin is inserted and linearly moves while the crank pin circularly moves; a linear guide rail which guides a linear movement of the linear slider; and a linear movement bar one end of which is connected to the linear slider and the other end of which is connected to the ultrasound transducer.

An opening may be formed to the housing and the linear movement bar may move through the opening.

A sealing cap may be provided to the opening in order to prevent the ultrasound transmitting medium from being leaked through the opening from the housing.

A portion of an outer surface of the housing may be enclosed by a cover and the driving motor, the crank arm, the crank pin, the linear slider and the linear guide rail may be disposed inside the cover.

The driving unit may further include a linear guider which guides a linear movement of the ultrasound transducer.

The linear guider may include: a supporter which connects the ultrasound transducer and the liner movement bar and is provided with a guide hole; and a guide rod of a linear shape which is elongated along a longitudinal direction of the housing inside the housing and is inserted into the guide hole.

The driving unit may further include a vibration preventing part which prevents a vertical vibration of the linear movement bar.

The vibration prevention part may include: an upper supporting member which is fixed to the fixing bracket and supports an upper portion of the linear movement bar; and a lower supporting member which is fixed to the fixing bracket and supports a lower portion of the linear movement bar.

The apparatus may further include a position control unit which controls a position of the ultrasound transducer.

The position control unit may include a position detector which detects a position of the ultrasound transducer.

The position detector may include: a magnetic body which is provided to a supporter which supports the ultrasound transducer; and a magnetic sensor which is provided to the housing or a portion fixed to the housing to cooperatively work with the magnetic body and detects a position of the magnetic body.

The ultrasound transducer may be located at an initial position, initial positions of the magnetic body and the magnetic sensor may be determined with reference to a focus of an ultrasound generated by the ultrasound transducer.

The position control unit may further include a memory stores a number of movement of the ultrasound transducer by the position detector.

The position control unit may further include a controller which controls a moving distance and a number of movement of the ultrasound transducer based on information of the position detector and the memory.

### Advantageous Effects

According to an embodiment of the present invention, since the ultrasound transducer automatically moves inside the housing by the driving unit, the ultrasound transducer can automatically moves to plural target area or along a long target without the user's manual movement of the housing, so convenience can be enhanced.

Further, according to an embodiment of the present invention, since the position of the focus of the ultrasound transducer can be precisely detected by the position detector, the focus of the ultrasound transducer can be precisely positioned at a target point.

### BRIEF DESCRIPTION OF DRAWINGS

FIG. 1 is a cross sectional view schematically showing an apparatus for generating a high intensity focused ultrasound according to an embodiment of the present invention.

FIG. 2 is a cross sectional view taken along a line II-II in FIG. 1.

FIG. 3 is a block diagram showing a position control unit and a driving motor of an apparatus for generating a high intensity focused ultrasound according to an embodiment of the present invention.

FIG. 4 is a cross sectional view schematically showing the state in which a ultrasound transducer of an apparatus for generating a high intensity focused ultrasound according to an embodiment of the present invention is moved.

FIG. 5 is a drawing which is seen in a different angle showing the state in which an ultrasound transducer of an apparatus for generating a high intensity focused ultrasound according to an embodiment of the present invention is moved.

### DETAILED DESCRIPTION OF THE EMBODIMENTS

Hereinafter, embodiments of the present invention will be described in detail with reference to the accompanying drawings.

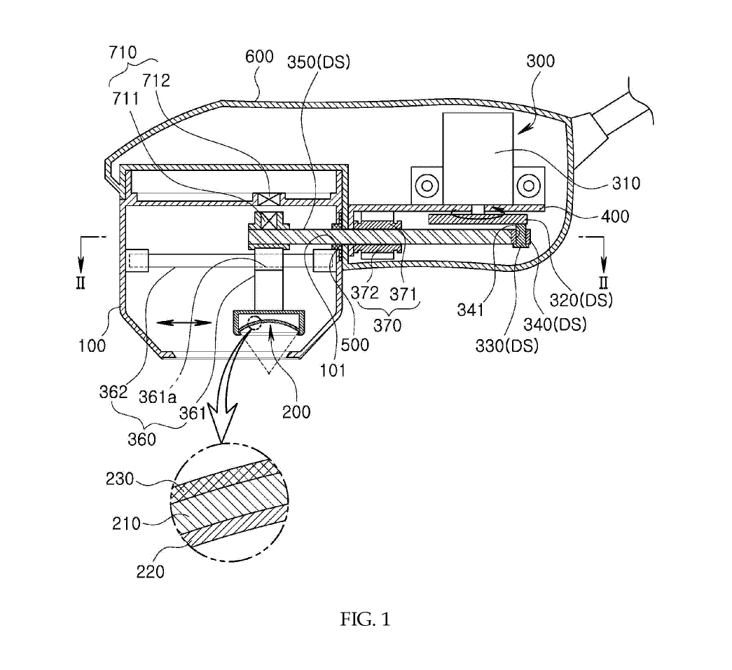

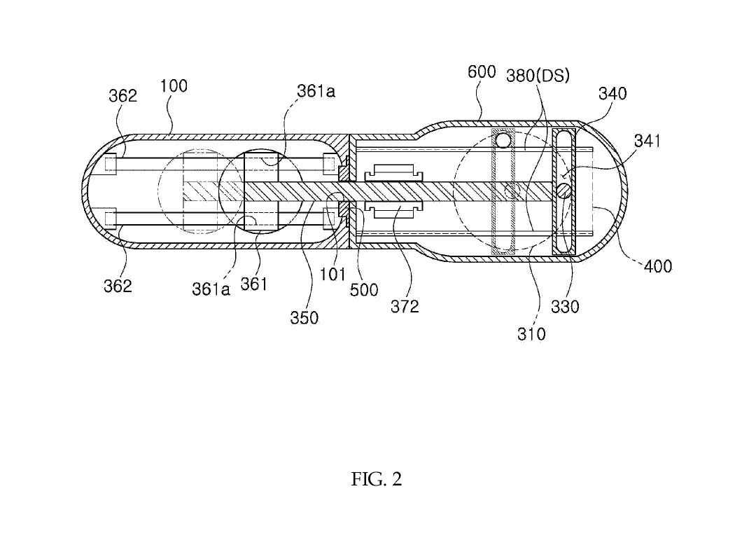

FIG. 1 is a cross sectional view schematically showing an apparatus for generating a high intensity focused ultrasound according to an embodiment of the present invention, and FIG. 2 is a cross sectional view taken along a line II-II in FIG. 1.

An apparatus for generating a high intensity focused ultrasound according to an embodiment of the present invention, as shown in FIG. 1 and FIG. 2, includes a housing 100, an ultrasound transducer 200 and a driving unit 300.

First, referring to FIG. 1, the housing 100 will be explained.

An inside space of the housing 100 is filled with an ultrasound transmitting medium (not shown) and may have a sealed structure for preventing the ultrasound transmitting medium from being leaked.

Further, the housing 100 has an opening 102 at a frontal end thereof, and a treatment window 110 may be provided at the opening 102. Accordingly, the ultrasound which is generated by the ultrasound transducer 200 passes through the treatment window 110 and may be then focused at an external one point.

Referring to FIG. 1, the ultrasound transducer 200 will be explained.

The ultrasound transducer 200 converts electrical signal to ultrasound and may be movably disposed inside the housing 100. Further, not shown in the drawings, the ultrasound transducer 200 may be electrically connected to the pulse power generator (not shown) via a cable (not shown). In more detail, the ultrasound transducer 200, as shown in FIG. 1, may include a piezoelectric member 210, a first electrode 220 and a second electrode 230. The ultrasound transducer 200 is a vibrator and may have a concave shape as a whole.

The piezoelectric member 210 may have a concave shape as a whole. The piezoelectric member 210 may be formed various materials such as ceramic, complex piezoelectric material, single-crystal quartz which can convert electrical signal to mechanical vibration. Further, the vibration frequency of the piezoelectric member 210 is generally determined according to the thickness thereof, and in the embodiment of the present invention, the range of this frequency is not limited and all vibration frequency which can be realized by the piezoelectric member 210 can be available. The vibration frequency of all ranges which can be used for ultrasound treatment can be included.

Further, the size of the ultrasound member 210 is not limited such that it can be suitable realized in accordance to the magnitude of the energy of the apparatus for generating high intensity focused ultrasound and the treatment purpose.

The first electrode 220 and the second electrode 230 may be formed to contact an inner surface (an inner surface of the concave shape and a surface which faces the focus) and an outer surface (an outer surface of the concave shape and a surface which is opposite to the surface facing the focus), respectively. For example, the first electrode 220 and the second electrode 230 may be formed of metal such as silver which has a good electrical conductivity.

The first electrode 220 and the second electrode 230 may electrically connected to the pulse power generator (not shown) to be applied with the pulse electricity generated by the pulse power generator (not shown). That is, the first electrode 220 may be electrically connected to one of a positive terminal and a negative terminal (or a ground terminal) of the output of the pulse power generator (not shown) via a first electrically conductive line (not shown), and the second electrode 230 may be electrically connected to the other one of a positive terminal and a negative terminal (or a ground terminal) of the output of the pulse power generator (not shown) via a second electrically conductive line (not shown). Accordingly, the pulse current generated by the pulse power generator (not shown) is applied to the first electrode 220 and the second electrode 230, and the applied pulse current flows through the piezoelectric member 210. If the current flows through the piezoelectric member 210, the piezoelectric member 210 vibrates by the piezoelectric effect of the same.

As described above, if the pulse current is applied to the first electrode 220 and the second electrode 230 so that the pulse current flows through the piezoelectric member 210, the piezoelectric member 210 vibrates, and this vibration has ultrasound characteristics and generates ultrasound in the ultrasound transmitting medium (not shown) enclosing the piezoelectric member 210, and this ultrasound propagates via the ultrasound transmitting medium (not shown) and is focused on one point.

Hereinafter, referring to FIG. 1 and FIG. 2, the driving unit 300 will be explained.

The driving unit 300 moves the ultrasound transducer 200 linearly, and may be realized as a direct linear moving type which directly moves the ultrasound transducer 200 using an actuator (not shown) such as a pneumatic cylinder or as an indirect linear moving type which indirectly moves the ultrasound transducer 200 by converting rotation motion of an output shaft of a driving motor 310. The indirect moving type suing the driving motor 310 (e.g., a step motor) may precisely control the movement. Hereinafter, the driving unit 300 of the indirect moving type will be explained in detail.

The driving unit 300, as shown in FIG. 1 and FIG. 2, may include a driving motor 310 and a direction converting part DS. The driving motor 310 may be fixed to the outside of the housing 100 by a fixing bracket 400. The direction converting part DS converts the rotation movement of the shaft of the driving motor 310 to the linear movement and provides linear movement force to the ultrasound transducer 200.

As an example, the direction converting part DS, as shown in FIG. 1 and FIG. 2, may include a crank arm 320 which is shaft-coupled to the driving motor 310, a crank pin 330 which is provided at an edge portion of the crank arm 320, a linear movement slider 340 which has a long slot 341 into which the crank pin 330 is inserted and undergoes linear movement during the circular movement of the crank pin 330, a linear guide rail 380 (in FIG. 2) which guides the linear movement of the linear movement slider 340, and a linear movement bar 350 one end of which is connected to the linear movement slider 340 and the other end of which is connected to the ultrasound transducer 200. As another example, not shown in the drawings, the direction converting part may have a rack-pinion structure.

In addition, an opening 101 may be formed in the housing 100, and in this case, the linear movement bar 350 may move toward the inside of the housing 100 or toward the outside of the housing 100 through the opening 101.

In addition, in order to prevent the ultrasound transmitting medium (not shown) from being leaked through the opening 101 from the housing 100, a sealing cap 500 may be provided to the opening 101. The sealing cap 500 may be formed of a flexible material such as rubber, and as shown in FIG. 2, may maintain sealing without hindering the movement of the linear movement bar 350.

In addition, in case that a portion of the outer surface of the housing 100 is enclosed by a cover 600, the driving motor 310, the crank arm 320, the crank pin 330, the linear movement slider 340 and the linear guide rail 380 may be disposed inside the cover 600, in order to improve the external appearance.

In addition, the driving unit 300 may further include an auxiliary linear guider 360 which guide the linear movement of the ultrasound transducer 200. As an example, the auxiliary linear guider 360 may include a supporter 361 which connects the ultrasound converter 200 and the linear movement bar 350 together and has a guide hole 361a, and a guide rod 362 of a linear shape which is elongated along a longitudinal direction of the housing 100 inside the housing 100 and is inserted into the guide hole 361. Accordingly, the ultrasound transducer 200 may maintain linear movement without shaking laterally or vertically by the auxiliary linear guider 360 while moving by the driving unit 300, so the apparatus for generating high intensity focused ultrasound can be precisely controlled.

In addition, the driving unit 300 may further include a vibration preventing part 370 which prevents vertical vibration of the linear movement bar 350. As an example, the vibration preventing part 370 may include an upper supporting member 371 which is fixed to the fixing bracket 400 and supports an upper portion of the linear movement bar 350 and a lower supporting member 372 which is fixed to the fixing bracket 400 and supports a lower portion of the linear movement bar 350. Accordingly, the linear movement bar 350 can be prevented from vibrating vertically while moving, so the apparatus for generating high intensity focused ultrasound can be precisely controlled.

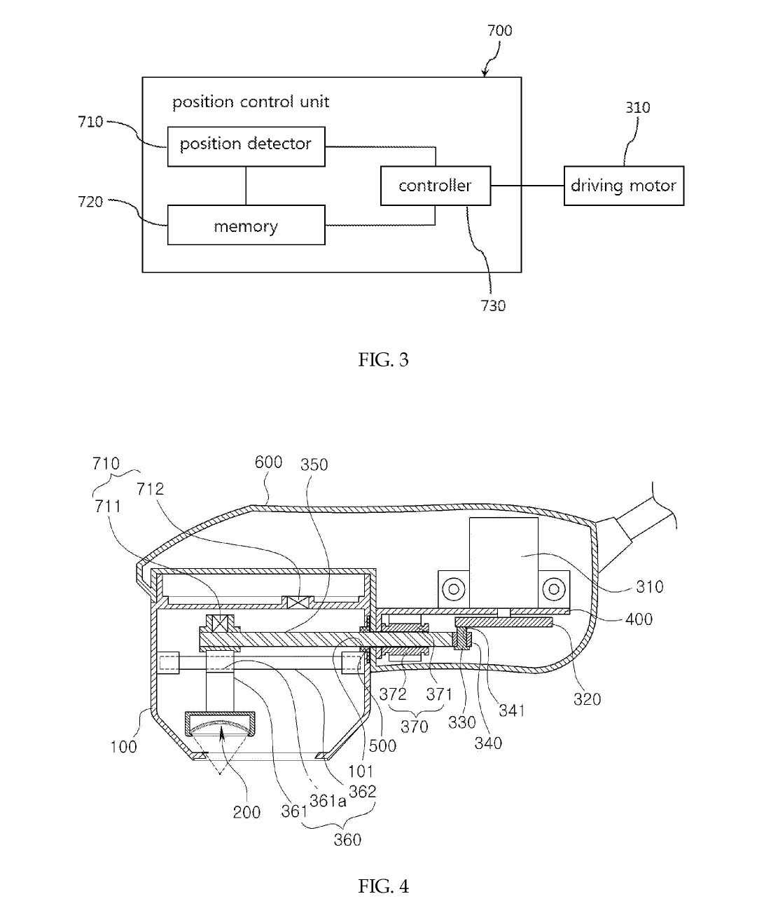

In addition, the apparatus for generating high intensity focused ultrasound may further include a position control unit 700. Hereinafter, referring to FIG. 3, the position control unit 700 will be explained in detail.

FIG. 3 is a block diagram showing a position control unit and a driving motor of an apparatus for generating a high intensity focused ultrasound according to an embodiment of the present invention.

The position control unit 700 controls the position of the ultrasound transducer 200 and may include a position detector 710 which detects the position of the ultrasound transducer 200.

The position detector 710 may include a magnetic body 711 which is provided to the supporter 361 which supports the ultrasound transducer 200 and a magnetic sensor 712 which is provided to the housing 100 or a portion (e.g., a printed circuit board (not shown) which is fixed to the housing 100 to work cooperatively with the magnetic body 711 and detects the position of the magnetic body 711. In particular, when the ultrasound transducer 200 is located at an initial position, initial positions of the magnetic body 711 and the magnetic sensor 712 may be able to be determined with reference to the focus of the ultrasound generated by the ultrasound transducer 200. By using the magnetic sensor, precise sensing is possible without being interfered by the ultrasound transmitting medium filled in the housing, compared to an infrared light sensor or the like.

Since the initial position is determined, the precise position of the ultrasound transducer 200 can be detected based on the number of the rotation of the driving motor 310, the distance between the center of the crank arm and the crank pin 330, and the like.

In addition, the position control unit 700 may further include a memory 720 which stores the number of the movement of the ultrasound transducer 200. Accordingly, the number of the movement of the ultrasound transducer 200 can be stored and it can be notified to a user.

In addition, the position control unit 700 may further include a controller 730 which controls the movement distance and the movement number of the ultrasound transducer 200 based on the information of the position detector 710 and the memory 720. Accordingly, if the suitable movement number is set depending on the size and the degree of the deceased part, the controller 730 sends a control signal to the driving motor 310 such that driving motor 310 operates the target number of the movement with reference to the precise initial position through the position detector 710.

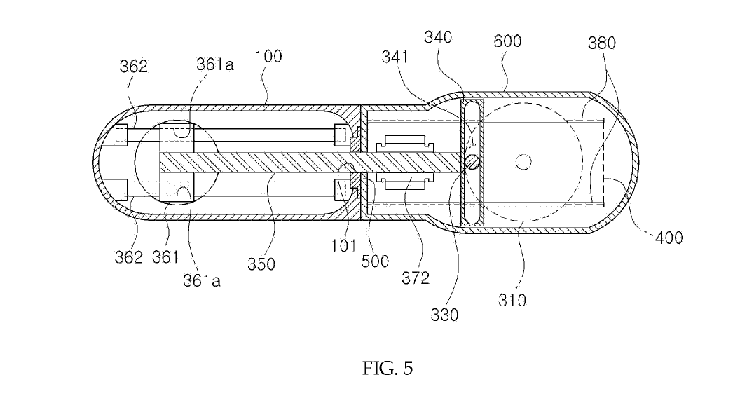

Hereinafter, referring to FIG. 1 and FIG. 5, the movement of the ultrasound transducer 200 of the apparatus for generating high intensity focused ultrasound according to an embodiment of the present invention will be explained in detail.

FIG. 4 is a cross sectional view schematically showing the state in which a ultrasound transducer of an apparatus for generating a high intensity focused ultrasound according to an embodiment of the present invention is moved, and FIG. 5 is a drawing which is seen in a different angle showing the state in which an ultrasound transducer of an apparatus for generating a high intensity focused ultrasound according to an embodiment of the present invention is moved.

First, if a control signal is transmitted to the driving motor 310 in a state of FIG. 1 and FIG. 2, the shaft of the driving motor 310 rotates in a counter clockwise to urge the crank arm 320 to rotate in a counter clockwise.

While the crank pin 330, which is fixed to the edge portion of the crank arm 320, rotates together while the crank arm 320 rotates, and the linear movement slider 340 into which the crank pin 330 is inserted and the linear movement bar 350, which is provided to the linear movement slider 340, move to the left in the drawing as shown in the dotted lines in FIG. 2.

While the linear movement bar 350 moves to the left, the linear movement bar 350 undergoes a linear movement along the auxiliary linear guide 360 to the left as shown in FIG. 3 and FIG. 4. During this process, the supporter 361 which is fixed to the linear movement bar 350 and the ultrasound transducer 200 which is fixed to the supporter 361 also move to the left in the drawing to treat the deceased portion. In particular, even when the deceased portion is long or when the deceased portions are plural, the deceased portion(s) can be treated while the ultrasound transducer 200 move linearly, and the treatment can be performed by adding the number of the movement of the ultrasound transducer 200.

The apparatus for generating high intensity focused ultrasound may have the following effects.

According to an embodiment of the present invention, since the ultrasound transducer 200 automatically moves inside the housing 100 by the driving unit 300, the ultrasound transducer 200 can automatically moves to plural target area or along a long target without the user's manual movement of the housing 100, so convenience can be enhanced.

Further, according to an embodiment of the present invention, since the position of the focus of the ultrasound transducer 200 can be precisely detected by the position detector 710, the focus of the ultrasound transducer 200 can be precisely positioned at a target point.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. An apparatus for generating high intensity focused ultrasound comprising:
a housing which is filled with ultrasound transmitting medium;
an ultrasound transducer which is movably disposed inside the housing; and
a driving unit which linearly moves the ultrasound transducer.

2. The apparatus of claim 1, wherein the driving unit comprises:
a driving motor which is provided at the outside the housing through a fixing bracket; and
a direction converting part which converts a rotation movement of a shaft of the driving motor to a linear movement and provides a linear movement force to the ultrasound transducer.

3. The apparatus of claim 2, wherein the direction converting part comprises:
a crank arm which is shaft-connected to the driving motor;
a crank pin which is provided at an edge portion of the crank arm;
a linear slider which has a long slot into which the crank pin is inserted and linearly moves while the crank pin circularly moves;
a linear guide rail which guides a linear movement of the linear slider; and
a linear movement bar one end of which is connected to the linear slider and the other end of which is connected to the ultrasound transducer.

4. The apparatus of claim 3, wherein an opening is formed to the housing and the linear movement bar moves through the opening.

5. The apparatus of claim 4, wherein a sealing cap is provided to the opening in order to prevent the ultrasound transmitting medium from being leaked through the opening from the housing.

6. The apparatus of claim 3, wherein a portion of an outer surface of the housing is enclosed by a cover and the driving motor, the crank arm, the crank pin, the linear slider and the linear guide rail are disposed inside the cover.

7. The apparatus of claim 3, wherein the driving unit further comprises a linear guider which guides a linear movement of the ultrasound transducer.

8. The apparatus of claim 7, wherein the linear guider comprises:
a supporter which connects the ultrasound transducer and the liner movement bar and is provided with a guide hole; and
a guide rod of a linear shape which is elongated along a longitudinal direction of the housing inside the housing and is inserted into the guide hole.

9. The apparatus of claim 7, wherein the driving unit further comprises a vibration preventing part which prevents a vertical vibration of the linear movement bar.

10. The apparatus of claim 9, wherein the vibration prevention part comprises:
an upper supporting member which is fixed to the fixing bracket and supports an upper portion of the linear movement bar; and
a lower supporting member which is fixed to the fixing bracket and supports a lower portion of the linear movement bar.

11. The apparatus of claim 1 further comprising a position control unit which controls a position of the ultrasound transducer.

12. The apparatus of claim 11, wherein the position control unit comprises a position detector which detects a position of the ultrasound transducer.

13. The apparatus of claim 12, wherein the position detector comprises:
a magnetic body which is provided to a supporter which supports the ultrasound transducer; and
a magnetic sensor which is provided to the housing or a portion fixed to the housing to cooperatively work with the magnetic body and detects a position of the magnetic body.

14. The apparatus of claim 13, wherein when the ultrasound transducer is located at an initial position, initial positions of the magnetic body and the magnetic sensor are determined with reference to a focus of an ultrasound generated by the ultrasound transducer.

15. The apparatus of claim 12, wherein the position control unit further comprises a memory stores a number of movement of the ultrasound transducer by the position detector.

16. The apparatus of claim 15, wherein the position control unit further comprises a controller which controls a moving distance and a number of movement of the ultrasound transducer based on information of the position detector and the memory.
